(19) United States
(12) Patent Application Publication
(10) Pub. No.: US 2007/0158456 A1
(43) Pub. Date: Jul. 12, 2007

(54) Long Term Active Fragrancer

(76) Inventor: Donald Spector, New York, NY (US)

Correspondence Address: Glen M. Diehl;DIEHL SERVILLA LLC, Suite 110, 77 Brant Ave., Clark, NJ, 07066, US

(21) Appl. No.: 11/621,379

(22) Filed: Jan. 9, 2007

Related U.S. Application Data
(60) Provisional application No. 60/757,352, filed on Jan. 9, 2006.

(51) Int. Cl. A24F 25/00; A61L 9/04

(52) U.S. Cl. 239/34; 239/38; 239/43; 239/51.5

(57) ABSTRACT

Method and apparatus for dispersing fragrances are disclosed. A fragrance emitting device includes a container having a reservoir and a liquid fragrance in the reservoir. A housing having a first and second opening is provided. The container is attached to a first opening in the housing in a position above the housing. A cap positioned on the second opening of the housing. The cap has an outlet covered with a semi-porous material. The liquid fragrance is delivered from the reservoir to the semi-porous material by gravity and is dispersed from the semi-porous material in vapor or gas form. A fan and/or a heater can be included to enhance the dispersement of the fragrance from the semi-porous material.

### CROSS-REFERENCE TO RELATED APPLICATIONS

This application is a utility application which claims the benefit and priority of U.S. Provisional Application No. 60/757,352, filed Jan. 9, 2006, the disclosure of which is hereby incorporated herein by reference thereto.

### BACKGROUND

This application relates to the field of fragrance systems and methods that actively disseminate fragrance over long periods of time.

There are many situations where unpleasant odors must be dealt with. Some of those situations always have unpleasant odors. Common examples are Industrial applications, such as rest rooms, or other public areas.

Many of these situations do not generally require variation in fragrance effect because the intended users would change regularly. Therefore the fragrance effect can be concentrated on the intended user's immediate reaction upon walking into a public area. Likewise, certain spaces in homes require a stronger fragrance effect in order to mask offensive or unwanted odors. Within these applications, a demand exists for fragrance systems with long term application and the capability of easy replenishment.

### SUMMARY

In the present invention, the fragrance system utilizes an inverted bottle containing a liquid fragrance. The liquid fragrance flows into a reservoir using gravity. In this respect it operates as a water cooler where there is no mechanical or electrical means necessary to continually supply the fragrance system with liquid fragrance other than gravity.

After flowing into the reservoir, the liquid fragrance then flows into a scent diffuser that is made out of a semi porous material, such as glass teslin, or certain types of plastic. The scent diffuser is capable of holding the fragrance in liquid form while allowing the liquid fragrance to permeate through the membrane so that the oils within the liquid fragrance are exposed on the surface. This scent diffuser is designed so that it extends out at a 90 degree angle from the reservoir. To increase the surface area of the scent diffuser, it might be flattened out or even have a donut hole in the middle, thereby maximizing the surface exposed to a fan or heating element.

The fan or heating element may be used beneath the scent diffuser, which remains constantly saturated as long as there is fragrance in the bottle. When the bottle is empty, which might be visualized by a transparent section in the housing, the bottle is replaced while the reservoir, scent diffuser and the housing remain in place.

This is a simple way of providing constant feeding to the scent diffuser, while at the same time allowing for a long term application of a fragrancer for home or commercial purposes.

There is provided, in accordance with the principles of the present invention, a fragrance emitting device having an open reservoir held in a housing unit, liquid fragrance in the reservoir and a scent diffuser which extends out at a 90 degree angle from the reservoir that receives the liquid fragrance through an opening in the reservoir. A heating element, fan or heating element and fan combination disperses the liquid fragrance from the scent diffuser in gas or vapor form using a battery power source, an electrical power supply or a solar power supply. In one aspect of the invention, the scent diffuser is a semi-porous membrane that is capable of holding the liquid fragrance so that it does not drip out of the reservoir. Further, in another aspect of the reservoir may be transparent so that the amount of liquid fragrance therein can be visualized.

In accordance with the present invention, a method of dispersing fragrance is also disclosed whereby the user fills the open reservoir with a liquid fragrance. The liquid fragrance is then dispersed onto a semi-porous membrane capable of holding the fragrance, so fragrance is prevented form dripping out of the reservoir. Thereafter, the liquid fragrance is evaporated from the semi-porous membrane into a gas or vapor using a fan, heating element or combination thereof.

In another aspect of the disclosed invention, a device is capable of receiving a liquid fragrance and emitting it in gas or vapor form. This device includes an open reservoir adapted to receive the liquid fragrance, a scent diffuser that can receive the liquid fragrance through an opening in the reservoir, a housing unit holding the reservoir and fan, heating element or combination thereof for dispersing the fragrance from the scent diffuser in a gas or vapor form. In one aspect of the disclosed invention, the scent diffuser is a semi-porous membrane extending out at a 90 degree angle from the reservoir. In another aspect of the invention, the scent is capable of holding the liquid fragrance, whereby the fragrance is prevented from dripping out of the cap.

### BRIEF DESCRIPTION OF THE DRAWINGS

FIG. 1 illustrates a view schematic of the long term active fragrancer in accordance with one aspect of the present invention.

FIG. 2 illustrates another view schematic of the long term active fragrancer in accordance with one aspect of the present invention.

FIG. 3 illustrates one embodiment of a container having a reservoir, a cap having a semi-porous material, a housing and means for enhancing dispersement of fragrances from the semi-porous material that can include a fan, a heating element or a fan and heating element combination.

FIGS. 4 and 5 illustrate a wall housing unit for the present invention.

Before describing several exemplary embodiments of the invention, it is to be understood that the invention is not limited to the details of construction or process steps set forth in the following description. The invention is capable of other embodiments and of being practiced or being carried out in various ways.

### DETAILED DESCRIPTION

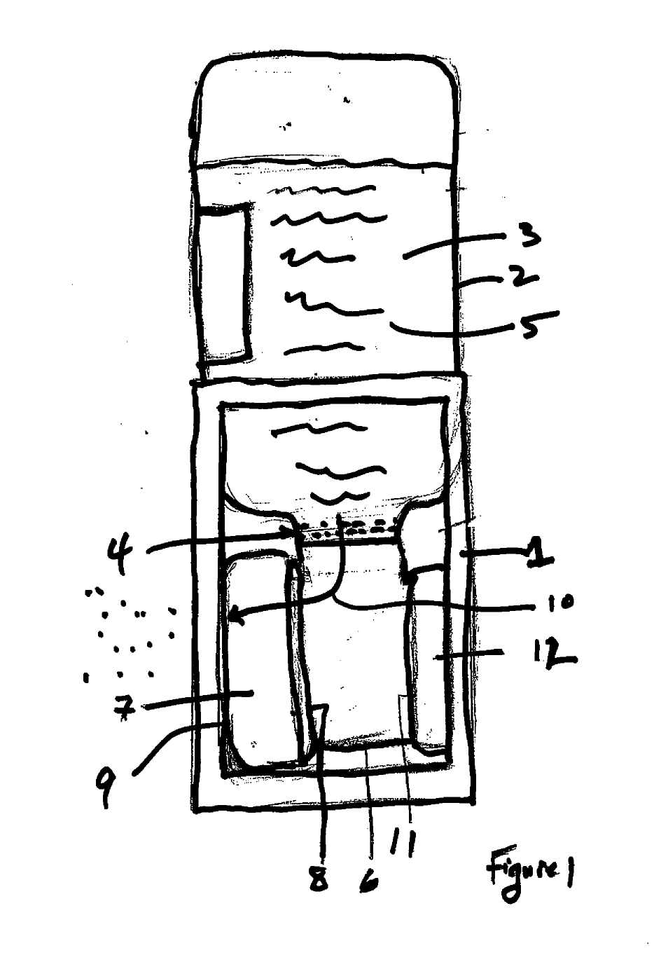

Referring now to FIG. 1, there is shown a view of one embodiment of a long term active fragrancer in accordance with the invention. A housing unit 1 holds a container 2 with a reservoir 3. The container 2 has a first end 4. The container 2 holds a liquid fragrance 5 in the reservoir 3.

The end 4 of the container 2 is connected to an opening in a housing 6. A cap 7 is connected to a second end 8 of the housing 6. The container 2 and the cap 7 are preferably threaded onto the housing 6. Other attachment means can also be used to connect the container 2 and the cap 7 to the housing 6.

The container 2 is preferably clear so that it is easy to determine how much liquid fragrance 5 is in the container 2.

In operation, the liquid fragrance 5 is fed by gravity into the housing 6 and then through the opening 8 into the cap 7. The cap 7 has a semi-porous material 9 on at least a part of an outlet in the cap 7. The liquid fragrance 5 is directed onto the semi-porous material 9 by the gravity, as indicated by arrow 10. The liquid fragrance 5 evaporates while on the semi-porous material 9 and is emitted as a gas or vapor to the surrounding environment.

The semi-porous material can be, for example, glass teslin or plastic. Other materials that are semi-porous to liquid fragrances can also be used.

The housing 6 can have a third opening 11. A means to enhance dispersement 12 can be connected to the housing 6. The device 12 can be a fan or a heater or both.

In the case of a fan, the device 12 would blow air onto the semi-porous material 9 to enhance the dispersement of vapor or gaseous fragrance from the material 9. In the case of a heater, the device would heat the liquid fragrance on the semi-porous material 9 to enhance the evaporation process.

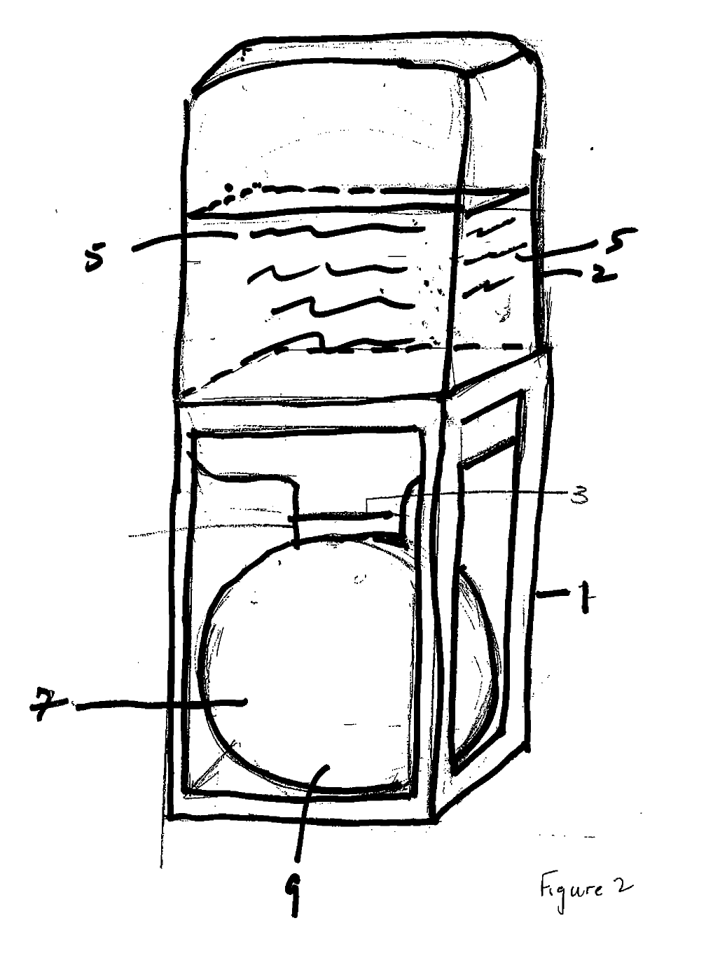

Referring now to FIG. 2, there is shown another view of one embodiment of the long term active fragrancer in accordance with the invention. In this view, the housing unit 1 holds the container 2. In alternative embodiments, the housing unit 1 can support the housing 6 instead. The container 2 can be any shape.

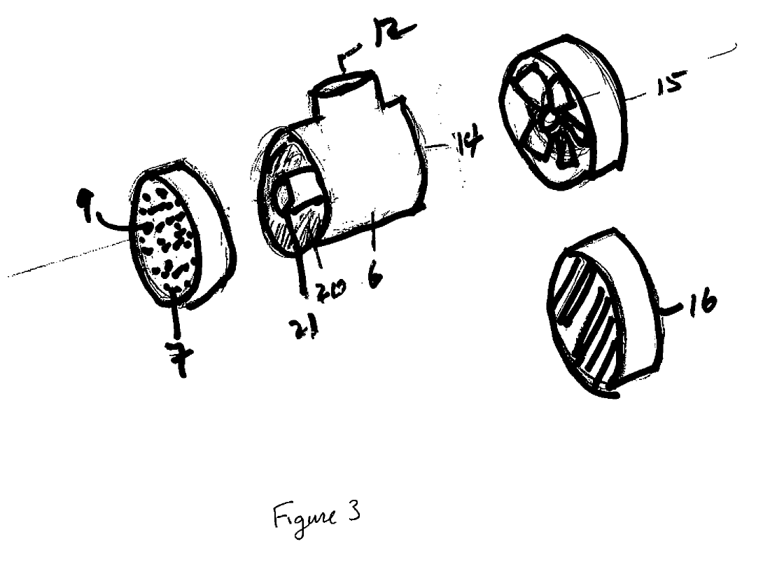

Referring now to FIG. 3, there is shown a schematic of a housing 6 in accordance with one aspect of the present invention. The housing 6 has three openings 12, 13 and 14. The first opening 12 is preferably threaded and receives the container 2. The second opening 13 is also preferably threaded and receives the cap 7 with the semi-porous material 9 on an outlet of the cap 7. The housing 6 can be provided with only the first and second opening 12 and 13.

Alternatively, a third opening 14 can also be provided. The third opening 14 is also preferably threaded and receives an attachment to enhance the dispersement of the fragrance. The attachment can be a fan 15 or a heater 16. The fan 15, in operation, blows air through the housing 6 through an area 20, while the fragrance is delivered through tube 21, to further enhance the dispersement of fragrance from the material 9. The heater 16, in operation disperses heat by the material 9 to further enhance the dispersement of fragrance from the material 9. Alternatively, both the fan 15 and the heater 16 can be attached to the housing 6.

The housing unit 1 may be partially enclosed so that the fragrance dispersed from the semi-porous material 9 on the cap 7 is circulated more efficiently. The housing unit 1 may be made of any material such as glass, metal or plastic. The material used should be capable of supporting the weight of the bottle 2. The size of the housing unit may also vary to accommodate different shaped and sized bottles.

The housing unit 1 may also be decorative and incorporate various design elements or ornamentation, including colored materials or fabric detail. The housing unit can also be configured so that the design elements can be easily changed. For example, the design elements can reflect seasonal or patriotic themes, holidays, or fictional children's characters.

In one embodiment of the disclosed invention, the housing unit constitutes a plurality of frames within which interchangeable slides may be placed. These slides may be opaque, transparent or semi-transparent and may include pictures, scenes, landscapes, abstract shapes, designs, words and numbers. The slides and/or housing unit may correspond to the characteristics of the liquid fragrance. For example, the fragrance may be of roses and the slide and/or housing unit may incorporate a bouquet of roses.

The container 2 may be made up of any fragrance resistant material, such as plastic, some metals, and glass. Further, the container 2 may be completely transparent or include a transparent section which allows the user to visually determine the amount of liquid fragrance remaining in the fragrancer. Similar to the housing unit, the reservoir may also be decorative and incorporate various design elements or ornamentation, including colored materials or fabric detail. The reservoir can be configured so that design element can be easily changed. For example, the design elements can reflect the seasonal or patriotic themes, holidays, or children's characters.

The semi-porous material 9 may include a semi-porous material such as glass teslin, or certain types of plastic that are capable of holding the liquid fragrance while allowing it to permeate through scent diffuser. The scent semi-porous material 9 should be shaped and sized to maximize the amount of exposed surface area. Preferably, the semi-porous material 9 is of the shape of shallow cylinder. It can also be of the shape of a cube or rectangle. The scent diffuser may also be of a variety of colors and textures. The colors may correspond with the housing unit or the liquid fragrance. Moreover, the scent diffuser may be textured in a manner to increase the surface area.

In certain embodiments, the cap 7 may also be decorative and incorporate various design elements or ornamentation, including color, seasonal or patriotic themes, holidays, or children's characters.

In one embodiment, the bottle 2 containing the fragrance is inverted and screwed into the housing 6 using the threads on the neck portion of the bottle, so that the bottle 2 is above the housing 6. Then, the fragrance 5 is fed by gravity through the housing 6 and the cap 7 onto the semi-porous material 9.

The bottle or container 2 should be made of a fragrance-resistant material such as plastic, glass or metal. The bottle can be molded to incorporate the design or decorative elements of the housing unit, reservoir or scent diffuser. Preferably, the bottle may include removable and interchangeable labels or covers which reflect the design or decorative elements of the housing unit, reservoir or scent diffuser.

In another embodiment, the end portion of the bottle opposite the neck portion may incorporate a vent. The vent can be a plastic lid that remains sealed until the user breaks the seal and opens the vent. Upon breaking the seal, air is allowed into the bottle through the opening. As air enters the opening, the liquid fragrance continually exits the bottle into the reservoir and finally into the scent diffuser. Alternatively, ventilation may be provided through the scent diffuser whereby ambient air enters the bottle through the scent diffuser and reservoir, flowing in the opposite direction of the liquid fragrance.

The fan 15, the heating element 16, and fan and heating element combination should be shaped and sized to enable attachment onto end 8 of the reservoir. They may be powered by any power source, such as battery power source, electrical power supply or a solar power supply.

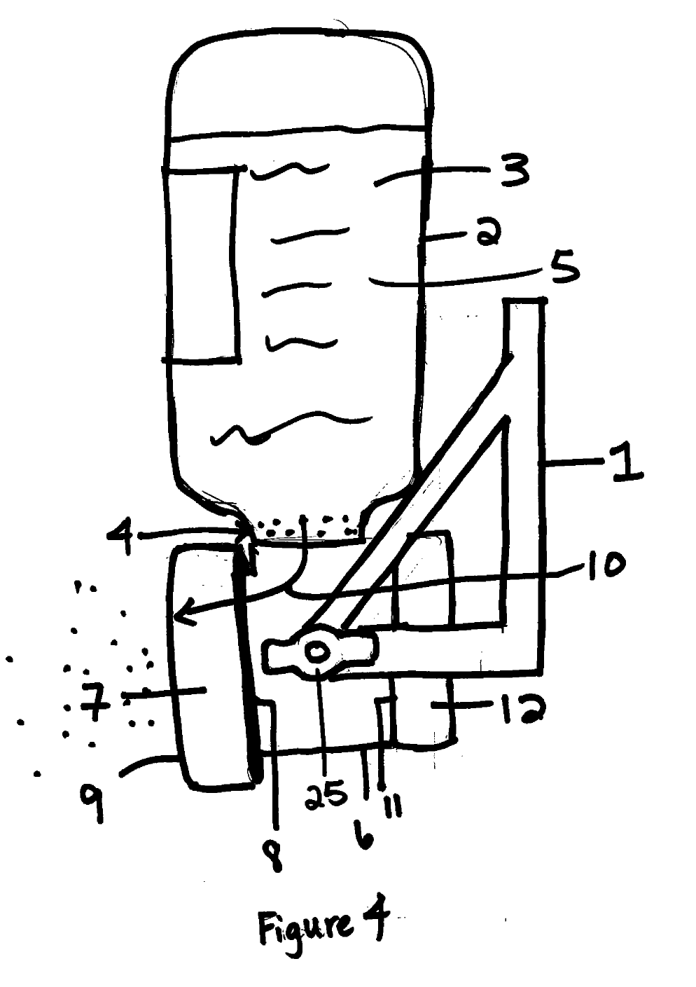

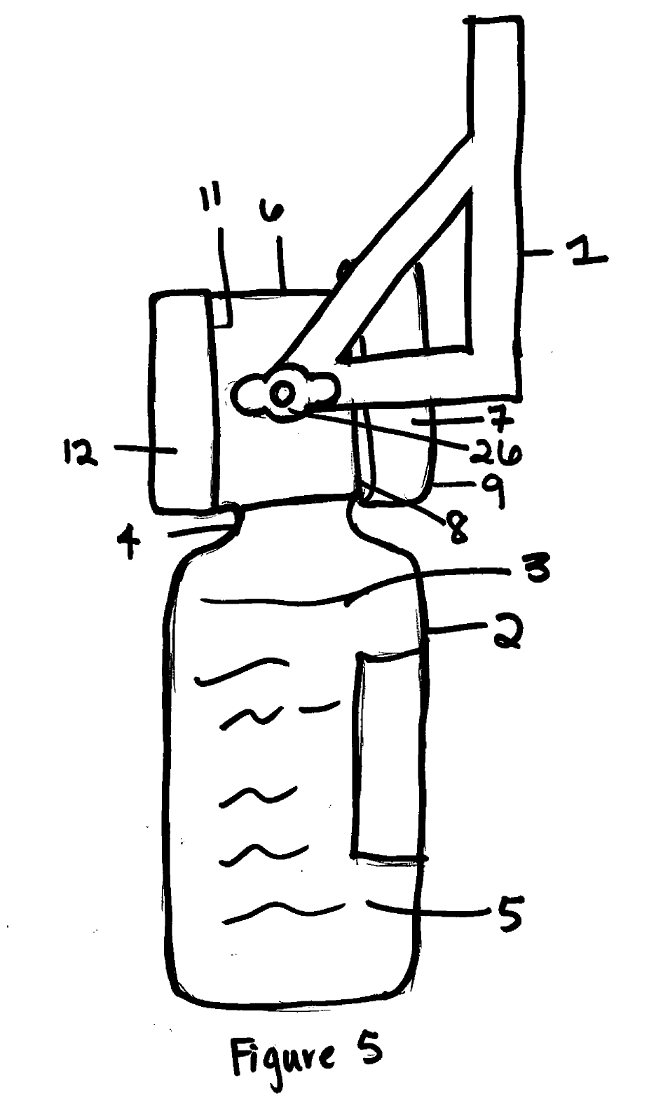

FIGS. 4 and 5 illustrate a wall housing unit for the present invention. The housing 6 is held in place by a wall housing unit 25. FIG. 4 illustrates a fragrance dispenser in accordance with one aspect of the present invention in operation. The container 2 is above the housing 6 so that fragrance is dispersed by gravity.

When the fragrance is gone and it is time to replace the container 2, the container 2 and the housing 6 are inverted so that the container 2 is below the housing 6, as shown in FIG. 5. Then any remaining fragrance in the container 2 cannot escape. The inverting of the container 2 can be accomplish, for example, by loosening two nuts 26 and 27 to relieve pressure on the housing 6. The container 2 and the housing 6 can then be inverted. While inverted, the container 2 is removed from the housing 6 and a new container 2 containing fragrance can be threaded onto the housing 6. The container 2 and the housing 6 can then be inverted back into the position illustrated in FIG. 4.

The container 2 can have a protective cover that prevents the fragrance from escaping. The cover can be removed before attachment of the container 2 to the housing 6 or the cover can be pierced when the container 2 is placed on the housing 6.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. A fragrance emitting device, comprising:
a container having a reservoir and a liquid fragrance in the reservoir;
a housing having a first and second opening, the first opening holding the container in a position above the housing; and
a cap positioned on the second opening of the housing; the cap having an outlet covered with a semi-porous material;
wherein the liquid fragrance is delivered from the reservoir to the semi-porous material by gravity.

2. The device of claim 1, further comprising means for enhancing dispersement of the liquid fragrance from the semi-porous material.

3. The device of claim 2, wherein the means for enhancing is connected to a third opening of the housing.

4. The device of claim 1, further comprising a fan that can project air near or on the semi-porous material.

5. The device of claim 1, further comprising a heater that can project heat near or on the semi-porous material.

6. The device of claim 4, further comprising a heater that can project heat near or on the semi-porous material.

7. The device of claim 1, wherein the semi-porous material is glass teslin or plastic.

8. The device of claim 1, wherein the reservoir is transparent and enclosed.

9. The device of claim 1, wherein the cap extends from the housing at a 90 degree angle.

10. The device of claim 1, wherein the container is threaded onto the first opening of the housing.

11. The device of claim 10, wherein the cap is threaded onto the second opening of the housing.

12. The device of claim 1, wherein the semi-porous material emits fragrance in a gas or a vapor form.

13. The device of claim 2, wherein the means for enhancing dispersement is powered using a power source selected from a group consisting of a battery power source, an electrical power supply and a solar power supply.

14. The device of claim 1, comprising a housing unit that holds the housing, the container and the cap that can be inverted to facilitate replacement of the container.

15. A method of dispersing fragrance, comprising:
placing a cap having a semi-porous material onto a first opening of a housing;
placing a container holding a liquid fragrance on a second opening of a housing such that the container is above the housing;
dispersing the liquid fragrance onto the semi-porous material by gravity evaporating the liquid fragrance on the semi-porous material into a gas or vapor.

16. The method of claim 15, wherein the step of evaporating the liquid fragrance uses a fan.

17. The method of claim 15, wherein the step of evaporating the liquid fragrance also includes a heating element.

18. The method of claim 17, wherein the step of evaporating the liquid fragrance uses a heating element.

19. The device of claim 1, wherein the semi-porous material is glass teslin.

20. The device of claim 1, wherein the semi-porous material is plastic.
